(19) United States
(12) Patent Application Publication
(10) Pub. No.: US 2019/0167478 A1
(43) Pub. Date: Jun. 6, 2019

(54) Device and Method for Axis Marking

(71) Applicant: Murad Sunalp, Tulare, CA (US)

(72) Inventor: Murad Sunalp, Tulare, CA (US)

(21) Appl. No.: 15/755,288

(22) PCT Filed: Aug. 25, 2016
(86) PCT No.: PCT/US2016/048785

§ 371 Date: Feb. 26, 2018

(51) Int. Cl. A61F 9/013; A61F 9/008

(52) U.S. Cl. CPC A61F 9/0136; A61F 9/008

(57) ABSTRACT

A method and device is provided for forming discernable markings within the cornea of an eye. Using a lens having a focal length to communicate and focus a laser to a focal point within the corneal layer of an eye, temporary markings can be formed within the corneal layer below its exterior surface. So formed the markings will remain for a number of days and provide the surgeon the ability to use the markings during eye surgery.

This application claims priority to U.S. Provisional Patent Application Ser. No. 62/209,734, filed on Aug. 25, 2015, which is incorporated herein in its entirety by this reference thereto.

### FIELD OF THE INVENTION

This invention relates in general to eye surgery. More particularly, it relates to a method generating a temporary visually-discernable mark on the eye of a patient, and devices adapted to impart such indicia or markings. The method and apparatus is employable to impart temporary indicia into the cornea for use in subsequent intraocular lens alignment and orientation during an implant thereof in the eye of a patient.

### BACKGROUND OF THE INVENTION

As lens implants for eyes, particularly astigmatic keratotomy and toric intraocular lens implantations, become more widespread, the accurate alignment of the implanted lens as positioned within the eye is a significant concern. Where there exists even a minor misalignment of the positioning of the lens within the eye, a significant portion of the corrective power is lost to the patient.

For example, a surgeon must be able to identify the steep axis which is needed for proper intraocular lens implant (IOL) alignment to achieve an optimum result. This is because for every 1° that an implanted toric IOL is off-axis, the lens ability for reducing astigmatism is decreased by 3.3%.

Consequently, a visually discernable axis reference to the surgeon showing a determined axis which may be employed during cataract and other eye surgery, as a guide line to rotate and align the implanted intraocular lens, is extremely important to avoid misalignment.

Conventionally, marking the eye of a patient to provide a visually discernable position reference is a significant potential source of error. Should the mark be misplaced, or as can easily occur where ink markings are used, should the mark disappear during surgery, resulting alignment of the implanted lens can be significantly impacted. As can the vision outcome to the patient.

While a number of high tech means of alignment have been developed which provide computer aided marking through a computer generated targeting, such is expensive to implement and not always accurate. Further, a majority of eye surgeons conventionally prefer to employ a simple felt-tip marker to place freehand markings on the eyeball of the patient to be used for alignment references. Using the imparted mark or markings, the surgeon can rotate the implanted lens to align with the markings of the eye. However, markers vary on width and can, thus, impart overly large markings on the eye which can be many degrees in width. Such markings yield diminished accuracy of the subsequent lens alignment since they are many degrees in width and placed on the eye freehand, which in itself can be inaccurate by a number of degrees.

Further, using a felt tip marker to impart ink to the eye can yield markings of highly variable duration on the eye itself. Should the imparted markings somehow disappear or smudge during the surgical procedure, a significant misalignment of the implanted lens can occur, thereby diminishing the visual correction to the patient.

As such, there exists an unmet need for a method and system for imparting indicia to form a temporary visually discernable mark on the eye of patients undergoing surgery. Such a system should impart a narrow, but highly visible position reference mark, in a location on the eye which is highly accurate to correlate to the axis thereof. Such a system and method should provide for one or a plurality of marks to be imparted in highly accurate positions on the eye, relative to its axis and to each other. Still further, such a system should provide such visually discernable marks, in a manner which does not require ink to be placed on the eye which as noted is inaccurate and easily erased at inopportune times. Finally, such a marking method and apparatus, should impart markings which once placed, will remain visually discernable and unmovable for at least a day and ideally two or more days to allow the marks to be imparted with extreme precision prior to the surgery to be performed, without fear the marks will disappear too soon.

The forgoing examples of related art and limitations related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### OBJECTS OF THE INVENTION

It is an object of this invention, to provide a method for imparting indicia to the cornea in a highly accurate fashion, which is not erasable, but which will naturally disappear within days.

It is an object of this invention to provide a system for imparting a visually discernable mark or marks upon the eye, in a highly accurate fashion, without inks or markers.

It is a further object of this invention to provide such markings which will remain visually discernable, and thereby employable for lens alignment, for at least two days.

It is a further object of this invention to minimize the potential for allergic or irritation reactions in the eyes of patients having their eyes marked for subsequent surgery.

These and other objects, features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### SUMMARY OF THE INVENTION

In accordance with the purposes of the present invention, as embodied and broadly described herein, the present invention provides a method and a number of devices employable in such a method, for imparting indicia to the tissue of corneal layer below the exterior surface. Such indicia provides visually discernable markings on the eyes of a patient, in extremely accurate positioning.

The markings, so formed by the method herein, while temporary, will remain visually discernable for one to two days. Consequently the method herein, allows for non-erasable precision marking of the eye in advance, which thereby allows surgery to be more easily scheduled and performed subsequent to the impartation of the marks on the eye surface, without concern for the marking being removed or otherwise being rendered inaccurate.

The corneal layer or layer forming the cornea, is composed of a number of distinct sections. An epithelium layer which is the surface layer of the cornea. The cells on this surface or epithelium layer, provide barrier function and a smooth surface for the tear film. Adjacent is situated the Bowman's layer or membrane, which is a tough layer of basement membrane right under the epithelium. Adjacent the Bowman's layer is the cornea stroma which is composed of highly arranged collagen fibers and supporting keratocytes. The cornea stroma provides the majority of the corneal layer. Adjacent the rear surface of the corneal layer is the Descemet's layer or membrane, which is important for the health of endothelial cells which form the endothelium at the rear of the corneal layer which functions as a barrier and a pump that keeps the cornea from getting too wet.

In the method herein, markings are formed within the corneal layer, a distance below the exterior surface defined by epithelium layer. Preferably the markings are formed between the Bowman's layer or membrane and the Descemet's layer or membrane, which is between 40 to 450 microns, below the corneal surface defined by the epithelium layer. As defined herein, the surface of the cornea is intended to be the epithelium layer, and the markings are positioned in a range between 40 to 450 microns below the cornea surface by employment of a YAG or equivalent directed energy device and a lens forming a focal point in the noted range. This forms temporary visually discernable indicia, or marks, upon the intrastromal cornea in one or a plurality of positions thereon. The indicia, so placed, provide one or a plurality of visually discernable marks to mark the corneal axis.

The indicia so positioned within the corneal layer of the eye, below the surface, renders a mark or markings which are subsequently employable by a surgeon during eye surgery, for example, for use in toric intraocular lens alignment during surgery. The formed marks are in the corneal eye tissue itself, and therefor will not move, fade, dissolve, or otherwise become invisible for one to two days.

Further, the indicia forms marks with extreme precision using the targeting component and YAG laser.

The marking component of one of the devices herein employable in the method herein, positions one or a plurality of lenses adjacent to the eye through which the beam of a YAG laser passes. The lens or lenses are formed to define a focal point for the laser beam, which is between 40 to 450 microns below the surface of the cornea, to form the indicia defining the markings within the corneal layer of the eye. Thus, the laser forms the markings within the corneal layer, below the surface, where they cannot accentually be removed, and where they will last for approximately two days before disappearing.

In one mode of the device enabling the method herein, the targeting component is engaged to rotate, which rotates the lens for focusing the laser immediately adjacent to the eyeball. This focuses the laser energy in a manner to place visually discernable marks within the cornea and also in a narrow width, and at a precise chosen radial position or positions around the pupil to further enhance accuracy.

As noted, in the method herein, a device herein may be employed during the imparting of the discernable mark or markings to the intrastromal cornea. In the method employing one mode of the device, an instrument is provided for the precise positioning within the layer of the cornea, in any radial position around the pupil of the eye, a temporary visually discernable mark. A preferred means of precisely targeting the beam from a Yag laser or similar device, is the disclosed instrument which includes the device with a lens which may be engaged to the distal end of a handle. The instrument having a targeting lens or lenses is positioned adjacent the eye to form markings employable to discern an axis and may also be positioned directly adjacent the eyeball. Once properly positioned, a yag laser is employed to impart the markings using the laser at, for example, 10 mjoules in a single shot at minimum reset level. The markings are thereby formed within the layer of the cornea, between 40 to 450 microns below its surface.

Intrastromal Yag focusing is done at the limbal cornea in targeted positions determined by the placement of the aperture or lens in the desired radial position around the pupil of the eye. Thereafter, 1 to 3 laser marks may be produced within the corneal tissue of the eye which are visually discernable and in fixed positions on the eye and relative to each other. The temporary, but fixed markings, will last for one to two days. The markings are employable for a subsequent intraocular lens alignment and orientation during an implant of such a lens into the eye of the patient.

In another preferred mode of the device enabling the method herein, the lens through which the directed energy, such as a YAG laser, is communicated to the eye surface is positioned on a central ring and may be rotationally engaged with the interior circumference of a surrounding exterior ring engaged to the handle. A center area of the central ring has an aperture therein and is employable for alignment with the pupil of the patient during use by the surgeon. A bubble level may be positioned within the central ring to allow the surgeon to level the central ring to aid in positioning. The exterior ring has indicia thereon radially positioned showing degree markers radially extending around the exterior ring.

At least one lens communicates through the central ring in-between the aperture and the rotational engagement with the interior circumference of the exterior ring. Rotation of the interior ring allows the surgeon to position the lens to any radial degree or position around the circular interior circumference of the exterior ring.

In this fashion the degree markers thereon are employable for determining the position of the lens radially and provide an extremely accurate target for the subsequent formation of a mark or marks in chosen position on the eye by communication of the laser or directed energy through the pre-positioned lens. In use with the patient's eye rendered numb, the aperture in the central area of the interior ring is aligned with the pupil and degree markers are aligned with 12 o'clock and 6 o'clock positions around the pupil or in positions desired by the surgeon. If provided, a bubble level is employable by the surgeon concurrently to aid in aligning the markers.

Thereafter, the lens portion may be rotated to a radial position around the interior circumference of the outer ring as desired to thereby place the lens as a target for the laser to position a mark on the surface of the eye of the patient. This proximate placement of the lens or lenses in a highly accurate targeting position allows for subsequent communication of the YAG laser therethrough or other means, to thereby form the mark on the surface of the eye through directed energy focused through the lens.

In another mode of the instrument employable for imparting a mark to the tissue of the eye of the patient which is temporarily stationarily positioned thereon, a plurality of lenses may be grouped in a position or positions on a central ring which is engaged with the interior circumference of an exterior ring which is operatively engaged with a handle. A center area of the central ring has an aperture therein which is employable for alignment with the pupil of the patient during use. As with the second mode of the instrument, the exterior ring has indicia thereon radially positioned showing degree markers radially extending around the exterior ring. Again, the bubble level can also be included as it has been found to aid in accurately positioning the axis markers.

A plurality of lenses communicates through the central ring in a plurality of positions all of which are in-between the aperture and the rotational engagement with the interior circumference of the exterior ring.

Rotation of the interior ring in its engagement with the exterior ring, allows the user to position any or all of the plurality of lenses to any position around the circular interior circumference of the exterior ring. In this fashion the degree markers thereon are employable for determining the position of the plurality of lenses.

The subsequent communication of the yag laser through the targeting lens or lenses, is communicated to a focal point within the corneal layer, which preferably is between 40-450 microns, below the exposed surface of the cornea. The lens or lenses, thus, provide for the formation of a plurality of marks on the eye in the targeted positions chosen, in the tissue of the underlying eye, where they will not move or erase, and will be visible for two days.

This high degree of accuracy of the size and positioning of the indicia forming the temporary marks is possible through the communication of the laser or directed energy through the plurality of lenses, which have been formed and which have been placed in targeting position, to communicate the beam of the laser therethrough to form the marks in the eye tissue.

In use with the patient's eye rendered numb, the aperture in the central area of the interior ring is aligned with the pupil and degree markers are aligned with 12 o'clock and six o'clock positions around the pupil or as desired. Thereafter, the plurality of lenses may be rotated to a determined radial target position around the interior circumference of the outer ring as desired. A subsequent communication of a YAG laser or other directed energy device adapted to the task, through the one or plurality of lenses placed in the chosen target positions, will form visually discernable marks into the tissue of the cornea of the underlying eye of the patient in the exact targeted positions determine by lens placement. In this and all modes of the device and method herein, the indicia, formed by the focusing of the laser below the surface of the cornea, defines discernable marks formed into the tissue of the eye of the patient. In all modes of the method and use of the devices herein, the formed marks are temporary but will remain discernable and in fixed positions for one to two days or more.

The instrument may be provided with external rings which are removably rotatably engaged with a plurality of replaceable interior rings. Such will allow for engagement of the interior ring of choice having one or a plurality of lenses formed therein, which are currently substantially between 110 and 140 diopters with 122 diopters being a current favorite. The lens or lenses so positioned, may then be rotated to a targeting position by rotation of the interior ring within the exterior ring with the instrument adjacent the eye of the patient.

Once the lens or lenses formed in the device, are set by the surgeon to the desired targeting position, the YAG laser or other directed energy device capable of forming the marks in the tissue of the eye, is communicated through the lens or lenses to make the desired markings. The markings as noted are formed into the corneal layer, between 40 to 450 microns, below the surface thereof.

Thereafter, for subsequent patients, the interior ring may be removed and replaced with another of choice having one or a plurality of lenses of the desired diopter communicating therethrough, for use in forming markings in the eye tissue of subsequent patients. In all modes of the instrument herein the outer ring may be formed of metal or plastic capable of exposure to temperatures used for sterilization without deformation such as stainless steel or titanium or high temperature resistant polymeric materials. The interior ring would be formed of one or a combination of materials from a group including plastic, glass, and metal.

With respect to the above description, before explaining at least one preferred embodiment of the herein disclosed method and device in detail, it is to be understood that the invention is not limited in its application to the details of construction and to the arrangement of the components in the following description or illustrated in the drawings. The device herein described and disclosed in the various modes and combinations is also capable of other embodiments and of being practiced and carried out in various ways which will be obvious to those skilled in the art.

Any such alternative configuration as would occur to those skilled in the art is considered within the scope of this patent. Also, it is to be understood that the phraseology and terminology employed herein are for the purpose of description and should not be regarded as limiting.

As such, those skilled in the art will appreciate that the conception upon which this disclosure is based may readily be utilized as a basis for designing other eye marking instruments and methods for positioning a discernable marking within the corneal layer of the eye tissue of a patient, and for carrying out the several purposes of the present disclosed device. It is important, therefore, that the claims be regarded as including such equivalent construction and methodology insofar as they do not depart from the spirit and scope of the present invention.

### BRIEF DESCRIPTION OF DRAWING FIGURES

The accompanying drawings, which are incorporated herein and form a part of the specification, illustrate some, but not the only nor exclusive examples of embodiments and/or features of the disclosed device. It is intended that the embodiments and figures disclosed herein are to be considered illustrative of the invention herein, rather than limiting in any fashion.

In the drawings:

FIG. 1 depicts the general steps of the method herein using a targeting instrument with a lens or lenses, in combination with a YAG laser, for forming discernable markings submerged in the corneal layer of the eye tissue of a patient.

FIG. 2 displays a depiction of an eye treated with the method herein, wherein at least one marking has been formed by and into the corneal layer thereof using the method and/or the instruments herein.

FIG. 2a, shows another depiction of an eye, where a plurality of markings are formed within the corneal layer of the eye, in radial positions defining an axis.

FIG. 2b depicts a sectional illustration of an eye, showing the formation of indicia defining markings within the corneal layer of the eye, below the surface.

FIG. 3 shows one mode of the instrument enabling the method herein, wherein a focusing lens is engaged to the distal end of a handle and is positionable over target positions of the eye to focus laser energy therethrough to form markings into the underlying eye tissue of the cornea below the surface.

FIG. 4 depicts a view of a mode of an instrument employable for imparting markings into the corneal layer of an eye showing a rotatable interior ring engaged to the interior circumference of an exterior ring engaged to a handle.

FIG. 5 is a sectional view through the instrument of FIG. 4 showing the clear plastic section which houses one or a plurality of lenses.

FIG. 6 depicts another mode of the instrument similar to that of FIG. 4 but having a plurality of aligned lenses communicating through the material forming the rotatable interior ring which will form markings within the corneal layer of tissue similar to those of FIG. 2a.

FIG. 7 shows a sectional view through the instrument of FIG. 6 depicting the aligned plurality of lenses.

FIG. 8 depicts a mode of the instrument herein having a targeting component engageable with a handle, and showing a plurality of lenses therein which focus the laser between 40 to 450 microns below the surface of the cornea.

FIG. 9 shows the device of FIG. 8, and also includes an optional bubble level for use during positioning upon or adjacent the eye of the patient.

FIG. 10 shows a mode of the device, wherein the lens focusing the layer below the surface of the cornea, is centered, and would be moved to position by the user.

FIG. 11 depicts a sectional view through the lens of FIG. 10.

FIG. 12 shows a targeting component adapted for engagement to a handle such as in FIG. 10, wherein the central portion is empty and has a bubble level positioned therein, and a plurality of lenses is positioned about the perimeter.

FIG. 13, is an example of one mode of a lens which is configured to make a focal point of the laser 0.3 mm or 300 microns below the surface of the cornea.

### DETAILED DESCRIPTION OF THE INVENTION

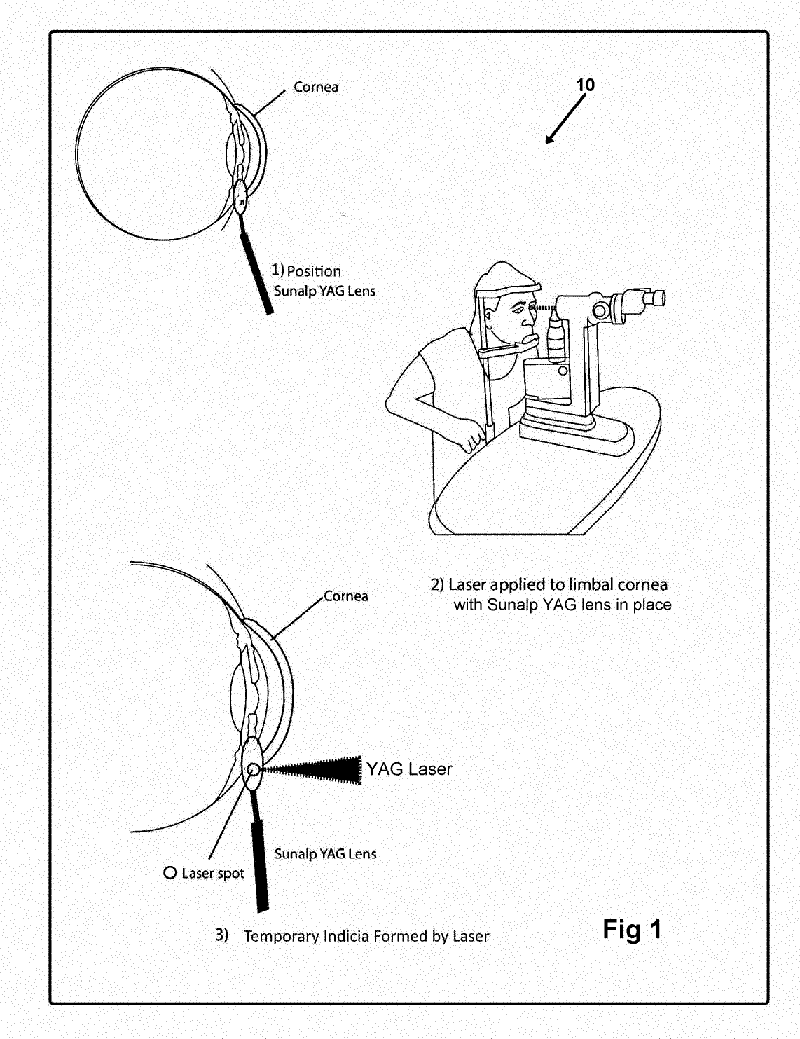

Now referring to drawings in FIGS. 1-13, wherein similar components are identified by like reference numerals, there is seen in FIG. 1, the steps in the method 10 herein disclosed for imparting markings into the cornea between 40-450 microns below the surface thereof. The formed temporary markings are subsequently employable for implanted lens orientation and will not move or erase for substantially two days.

In all modes of the method, at least one lens 16 for targeting the laser is positioned adjacent or upon the eye of the patient in a position where a marking 12 is desired. Once so positioned, in a next step, a YAG laser or other directed energy device is communicated through the targeting lens 16 and is focused to a focal point between 40 and 450 microns, below the surface of the cornea, within the corneal layer of the eye. The laser communicating through the lens 16 forms the markings 12 at the depth of and in a position within the cornea, determined by the lens 16 and its position on or adjacent the eye.

As noted, the formed markings 12, are temporary, but will be positioned viewable within the cornea, for substantially two days, so they are not subject to erasure or movement.

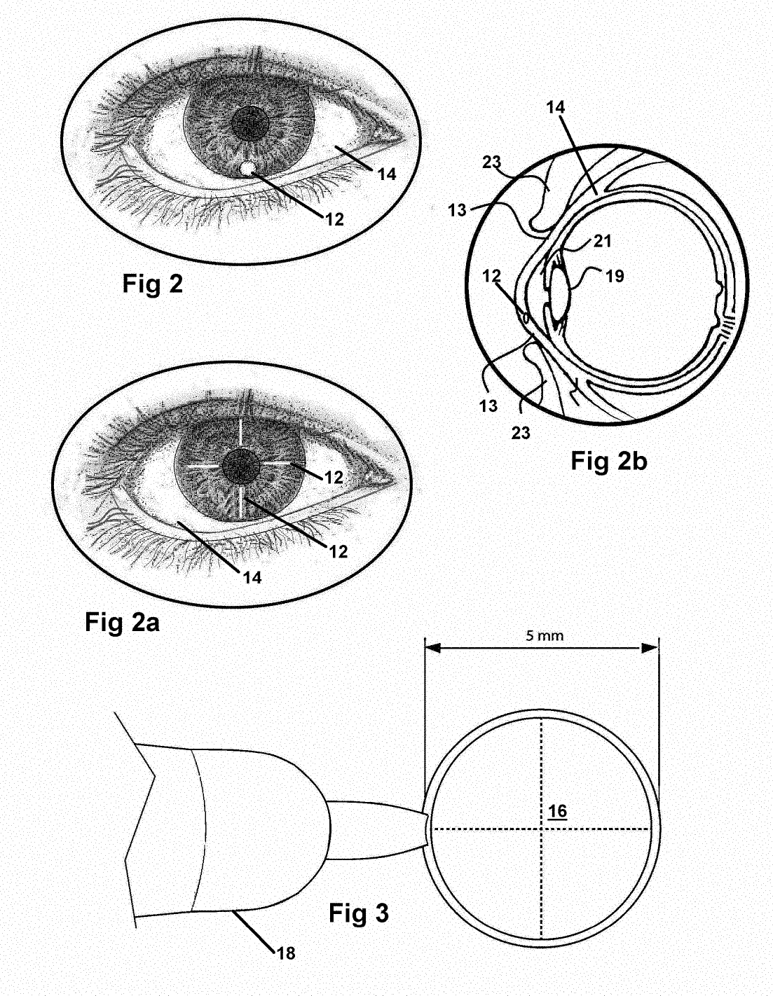

In a preferred mode of implementation, at least one marking 12 is formed into the corneal layer of the eye 14, as shown in FIG. 2, using the targeting instrument herein having at least one lens 16 configured to target the location of communication of the laser into the corneal layer of the eye. Multiple markings 12 may also be employed. While various devices are shown herein to accomplish this method, others are possible and considered within the scope of this application so long as they employ the method herein, of positioning markings 12 within the cornea between 40 to 450 microns below the surface thereof.

Markings 12 formed in the cornea are shown in FIGS. 2-2b herein. As shown in FIG. 2, an eye 14 is depicted which has been treated with the method 10 herein. As shown in FIG. 2, at least one marking has been formed by and into the corneal layer and is visible. Shown in FIG. 2a is another depiction of an eye 14, where a plurality of markings 12 are formed within the corneal layer of the eye 14, in radial positions.

In FIG. 2b, there is seen a sectional illustration of an eye 14. The marking 12 shown, as with all markings 12 formed in the method herein, is formed within the layer of the cornea 13, between 40 to 450 microns, away from or below the surface thereof. Also shown, as a frame of reference, are the eye lens 19, the area of the eye 14 of the iris 21, and the eyelids 23.

FIG. 3 shows one mode of the instrument for the method 10 herein wherein a glass or plastic lens 16 is engaged to the distal end of a handle 18 and is positionable to place the lens 16 over determined target positions of the eye 14. So positioned, the lens 16 has surfaces formed to focus the beam of laser energy therethrough to a focal point below the surface of the cornea 13 to form markings 12 into the underlying eye tissue. The lens 16 can be ground or formed to an exact focal point within the cornea 13 in the noted range, taking into consideration the distance from the lens 16 of the laser. An example of such a formation yielding a focal point 300 microns below the surface of the cornea 13 is depicted in FIG. 13. However, such focal point lens formation is well known and the lens 16 configuration can be changed to form a focal point anywhere between 40 to 450 microns below the surface of the cornea 13, and such is anticipated within the scope of this patent.

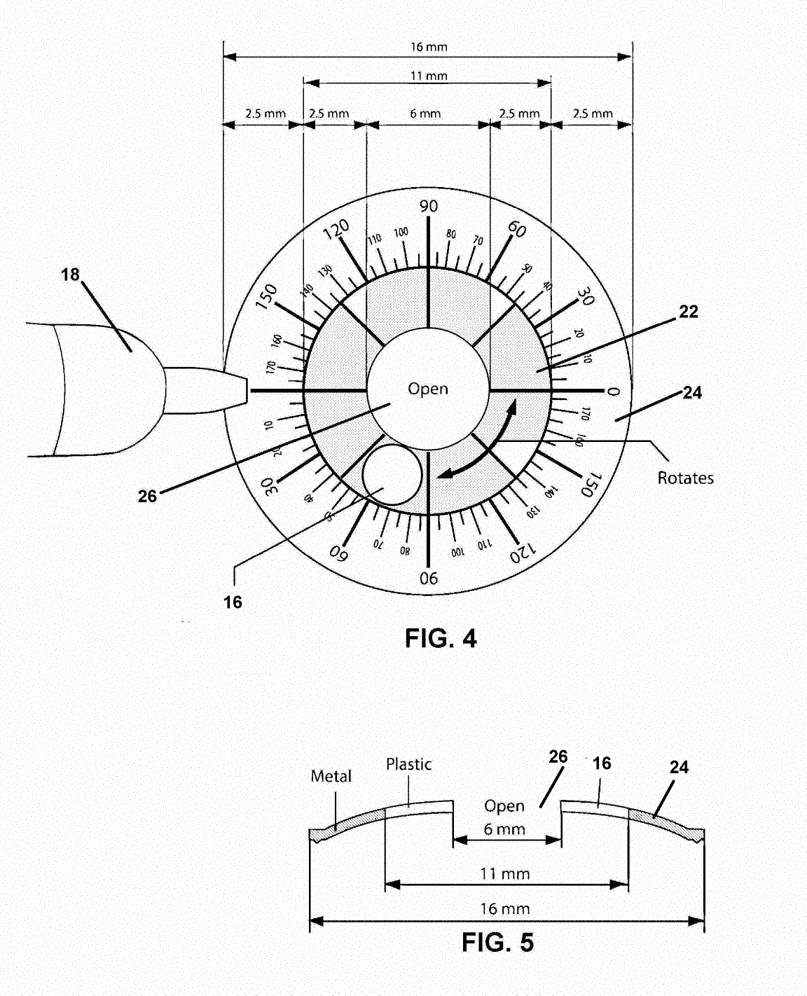

Another mode of an instrument having a lens 16 employable for imparting markings to the tissue of an eye 14 is depicted in FIGS. 4 and 5. As shown, a carrier is formed for the lens 16 by a rotatable ring 22 which is engaged to the interior circumference of an exterior ring 24 engageable to a handle 18. An aperture 26 is positioned in a center of the lens carrier formed by the ring 22 for use during alignment with the pupil of the eye 14.

A lens 16 communicates through the plastic or metallic material forming the lens carrier shown as the ring 22. The lens is rotatable by rotation of the ring 22 to position it in the desired target position for communication of a yag laser or other directed energy device adapted for forming the markings 12 into eye tissue. Indicia showing degrees is radially positioned around the surface of the exterior ring 24. In another step in the method 10 herein, an LED or other light source (not shown) may be employed during targeting of the lens 16 or lenses 16 herein, to determine the contact point of the light through the lenses on exterior of the cornea, prior to communication of laser energy to form the marking 12 below the surface of the cornea 13 and make the mark 12 on the targeted position.

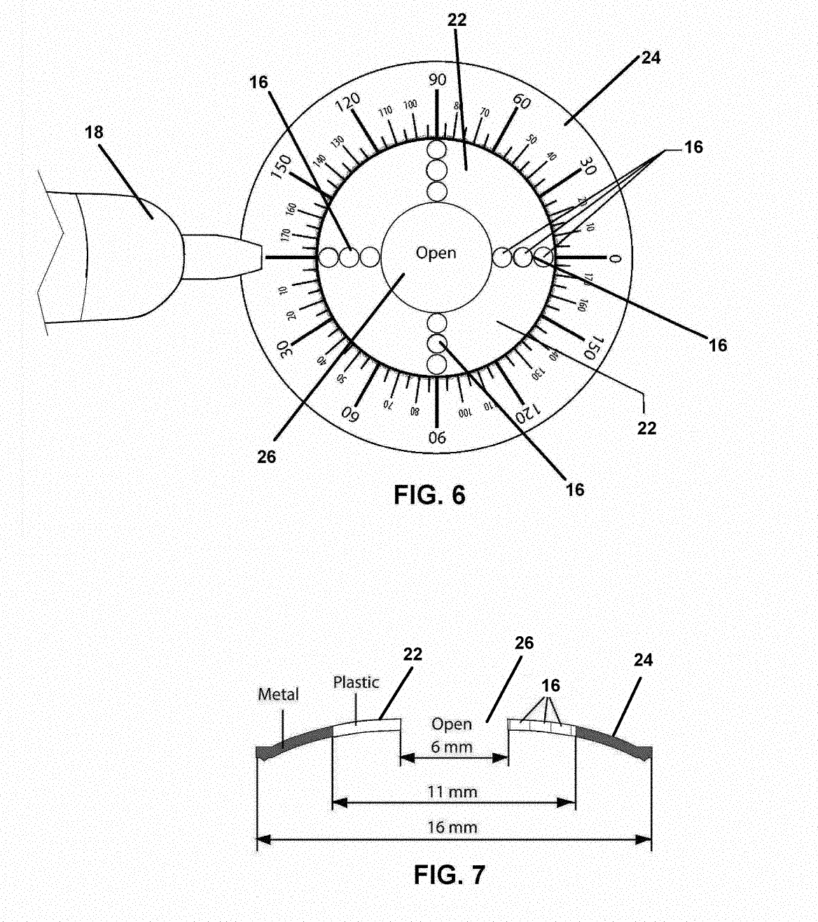

Shown in FIG. 6, is a mode of the instrument similar to that of FIG. 4. The instrument of FIG. 6 is shown having a plurality of lenses 16 communicating through the carrier formed by the material of the rotatable ring 22. The plurality of lenses 16 may be in a single group or multiple groups of lenses 16 as shown or in a plurality of singular lenses 16 communicating in different positions through the ring 22 which would form a line of markings.

As with the instrument of FIG. 4, the instrument of FIG. 6 preferably has the lens carrier shown as the ring 22 rotationally engaged with the exterior ring 24 so that it may be rotated and the lens 16 or lenses 16 thereby may be positioned adjacent degree markers radially positioned on the surface of the exterior ring 24, to determine the targeted positions for the markings 12.

The YAG laser or a similar directed energy device is employed subsequent to the final positioning of the lens 16 or lenses 16. By communication of a YAG laser beam therethrough, the formation of the fixed positioned temporary markings 12 within the layer of the cornea 13, at a depth and position targeted by the focal point of the lens 16.

These marks or markings 12 are visually discernable for adjustment or alignment of an implanted lens. As noted above, in a step of the method 10, and in combination with the devices herein, an LED or other light emitting device might first be employed to communicate a light beam through the lens 16 first, to ascertain the focal point of a lens 16 on the exterior surface of the cornea 13, to aid in placing the lenses 16 in determined targeting positions for placement of subsequent marks.

As noted, in all modes of the instrument with rotatable lenses 16, the lens carrier or ring 22 may be removably engageable with the exterior ring 24 to allow removal and replacement thereof with a chosen ring 22 having the chosen lens 16 or lenses 16 positioned therein, of a diopter and in positions adapted to form the markings 12 at the desired depth in the cornea 13 layer.

Thereafter, the ring 22 may be removed and replaced with another adapted for engagement within the interior circumference of the exterior ring 24. In this manner the lens carrier provided by the rings 22 may be provided in sterile packaging and in and infinite number of lens configurations, number, and positionings configured to form the markings 12 which are adapted to allow for alignment and adjustment of an implanted lens subsequently implanted in the eye of a patient having the markings 12 thereon.

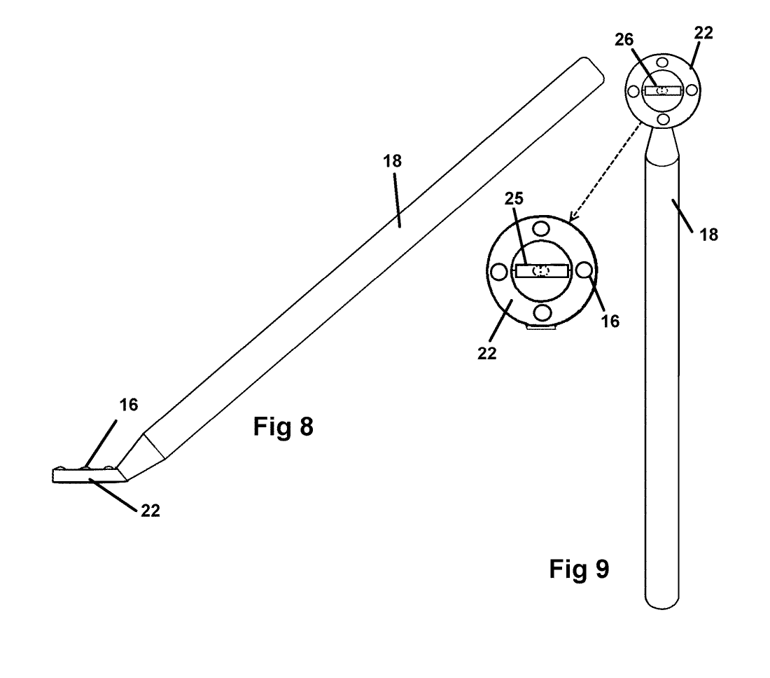

Shown in FIG. 8, is Amode of the instrument employable for the method 10 herein, having a lens carrier, engageable with a handle, and showing a plurality of lenses 16 therein which focus the laser between 40 to 450 microns below the surface of the cornea.

FIG. 9 shows the device of FIG. 8, and also includes an optional bubble level 25 for use during positioning of the carrier provided by the ring 22 and lens 16 upon or adjacent the eye 14 of the patient. As shown, the ring 22 is engaged or engageable with the distal end of a handle 18. A aperture 26 forming an open area in the center of the ring 22 has a bubble level 25 therein.

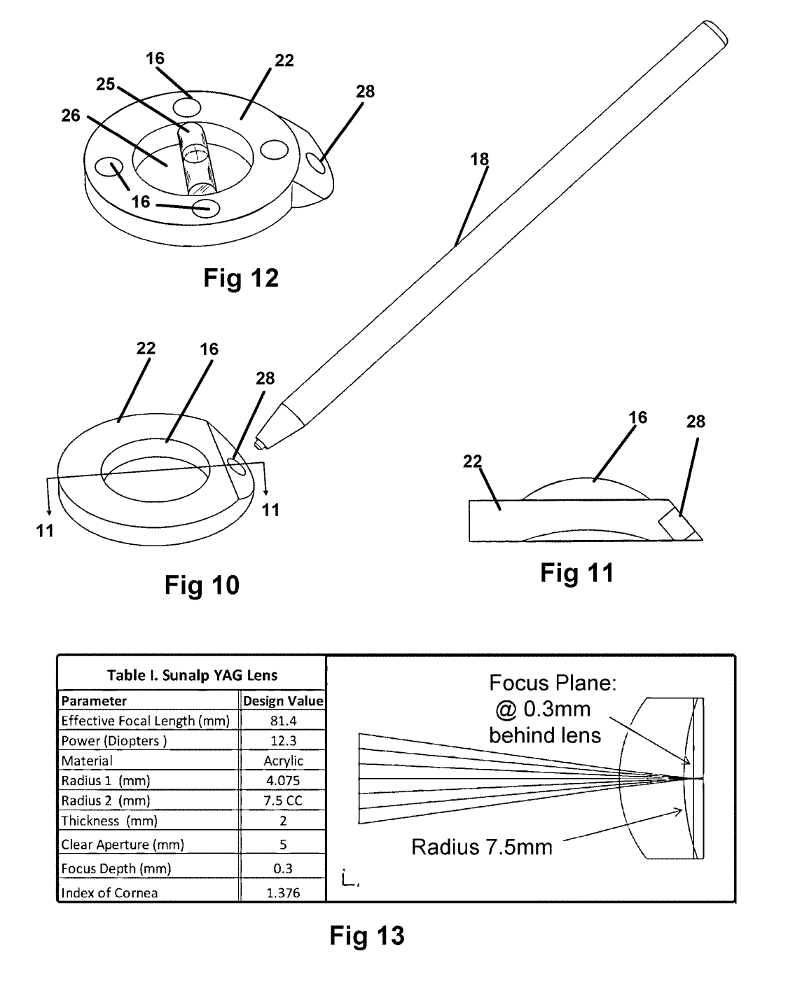

In FIG. 10 is depicted another mode of the instrument employable with the method 10 herein. As shown, the lens 16 for focusing of the laser to form the marking 12 below the surface of the cornea 13, is formed centrally within the ring 22 providing a lens carrier. In this mode, the lens 16 is moved to the area of the cornea 13 generally outside the pupil or over the iris, whereupon the laser is employed to form the marking 12 between 40 to 450 microns from the surface of the cornea 13 but within the layer formed by the cornea 13.

The ring 22 is shown as having a connection 28 thereon adapted to engage the distal end of a handle 18 to allow for engagement of any of a plurality of rings 22 having one or a plurality of lenses 16 positioned to form the marking 12 or markings 12 desired in the cornea 13 layer. A sectional view of the ring 22 is shown in FIG. 11.

Shown in FIG. 12 is another mode of the instrument herein showing the lens carrier provided by the ring 22 having a plurality of four lenses 16 therein and an aperture 26 communicating through the center. A bubble level 25 is shown positioned in the aperture 26, and a connection 28 is provided which is adapted to engage the distal end of a handle 18 of choice.

Finally, shown in FIG. 13, is an example a construction of a lens 16, which is configured to form a focal point of the laser below the surface of the cornea 13 and within the layer formed by the cornea 13. As shown, a lens 16 is configurable to cause the laser communicated therethrough, to have a focal point at substantially 0.3 mm or 300 microns, below the surface of the cornea 13, and within the layer of the cornea 13. A current particularly favored position for the marking 12 within the layer of the cornea 13 is between 200 to 400 microns below the exterior surface of the cornea 13 as such has shown to provided laser-formed markings 12 which are especially easy to view and discern.

Using lens grinding calculations which are well known, this focal point may be adjusted and in the method 10 herein, a lens 16 configured to position the focal point of the laser between 40 to 450 microns below the exterior surface of the cornea 13, and thereby form the temporary markings 12 at that position in the corneal layer 13 is preferred to place the markings 12 in position in the corneal 13 layer, where they are viewable by the physician, but where they cannot be accidentally removed or shifted.

As noted, any of the different configurations and components shown herein, can be employed with any other configuration or component shown and described herein. Additionally, while the present invention has been described herein with reference to particular embodiments thereof, and steps in the method, a latitude of modifications, various changes and substitutions are intended in the foregoing disclosures, it will be appreciated that in some instance some features, or configurations, or steps in formation of the invention could be employed without a corresponding use of other features without departing from the scope of the invention as set forth in the following claims.

All such changes, alternations and modifications as would occur to those skilled in the art are considered to be within the scope of this invention as broadly defined in the appended claims.

Further, the purpose of any abstract of this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

What is claimed is:

1. A method for forming discernable markings within the cornea of an eye, comprising the steps of:
positioning a lens having focal length to converge a laser communicating therethrough, to a focal point within the corneal layer of the eye, adjacent a surface of the cornea of said eye; and
communicating a laser beam through said lens to thereby form a marking at said focal point of said lens, within the corneal layer of the eye.

2. The method according to claim 1 further comprising the steps of:
positioning said lens having a said focal length defining a said focal point which is in a range between 40 to 450 microns below said surface of said cornea.

3. The method according to claim 2, further comprising the steps of:
positioning a plurality of said lenses to form a plurality of markings within said corneal layer.

4. An apparatus for forming discernable markings in an eye, comprising:
a lens carrier, said lens carrier having at least one lens therein;
said lens carrier adapted for positioning to a used position, adjacent the surface of a corneal layer of an eye;
said lens having focal length configured to converge a light communicating therethrough, to a focal point within said corneal layer a distance below said surface of said corneal layer; and
a YAG laser, said YAG laser positionable to communicate a laser beam through said lens to said focal point, whereby a marking is formed by said laser beam at said focal point.

5. An apparatus for forming discernable markings in an eye of claim 4, additionally comprising:
said lens carrier having a plurality of lenses positioned therein, wherein a plurality of said markings are formed within said corneal layer.

6. An apparatus for forming discernable markings in an eye of claim 4, wherein said lens has a said focal length configured to converge a said light communicating therethrough, to a said focal point within said corneal layer in a range from 40 to 450 microns below said surface of said corneal layer of said eye.

7. An apparatus for forming discernable markings in an eye of claim 5, wherein each said lens has a said focal length configured to converge a said light communicating therethrough, to a said focal point within said corneal layer in a range from 40 to 450 microns below said surface of said corneal layer of said eye.

8. The apparatus for forming discernable markings in an eye of claim 4 wherein said lens carrier has a connection thereon, said connection adapted to engage with a distal end of a handle.

9. The apparatus for forming discernable markings in an eye of claim 5 wherein said lens carrier has a connection thereon, said connection adapted to engage with distal end of a handle.

10. The apparatus for forming discernable markings in an eye of claim 6 wherein said lens carrier has a connection thereon, said connection adapted to engage with a distal end of a handle.

11. The apparatus for forming discernable markings in an eye of claim 4 additionally comprising:
said lens carrier rotationally engaged within an outer ring;
said lens rotatable to differing positions adjacent said surface of said corneal layer of said eye, by a rotation of said lens carrier within said outer ring.

12. The apparatus for forming discernable markings in an eye of claim 5 additionally comprising:
said lens carrier rotationally engaged within an outer ring;
said lens rotatable to differing positions adjacent said surface of said corneal layer of said eye, by a rotation of said lens carrier within said outer ring.

13. The apparatus for forming discernable markings in an eye of claim 6 additionally comprising:
said lens carrier rotationally engaged within an outer ring;
said lens rotatable to differing positions adjacent said surface of said corneal layer of said eye, by a rotation of said lens carrier within said outer ring.

14. The apparatus for forming discernable markings in an eye of claim 7 additionally comprising:
said lens carrier rotationally engaged within an outer ring;
each of said plurality of lenses being rotatable to differing positions adjacent said surface of said corneal layer of said eye, by a rotation of said lens carrier within said outer ring.

15. The apparatus for forming discernable markings in an eye of claim 11 additionally comprising:
said outer ring having a connection thereon, said connection adapted to engage a distal end of a handle.

16. The apparatus for forming discernable markings in an eye of claim 12 additionally comprising:
said outer ring having a connection thereon, said connection adapted to engage a distal end of a handle.

17. The apparatus for forming discernable markings in an eye of claim 13 additionally comprising:
said outer ring having a connection thereon, said connection adapted to engage a distal end of a handle.

18. The apparatus for forming discernable markings in an eye of claim 14 additionally comprising:
said outer ring having a connection thereon, said connection adapted to engage a distal end of a handle.
